(19) United States
(12) Patent Application Publication
(10) Pub. No.: US 2012/0157905 A1
(43) Pub. Date: Jun. 21, 2012

(54) METHODS FOR TREATING CARBON MONOXIDE POISONING BY TANGENTIAL FLOW FILTRATION OF BLOOD

(75) Inventor: Lakshman R. SEHGAL, Dana Point, CA (US)

(73) Assignee: BIOVEC TRANSFUSION, LLC, Chicago, IL (US)

(21) Appl. No.: 13/290,262

(22) Filed: Nov. 7, 2011

Related U.S. Application Data
(60) Provisional application No. 61/423,176, filed on Dec. 15, 2010.

(51) Int. Cl. A61M 1/34; B01D 61/00

(52) U.S. Cl. 604/5.04; 210/640

(57) ABSTRACT

The present application relates to a system and method for treating carbon monoxide poisoning. The system and method comprises passing the blood of a subject in need of such treatment through a filter unit comprising a gas permeable membrane to remove carbon monoxide from the blood by filtration against an extraction fluid enriched with oxygen and/or by exposure to one or more agents suitable for removing carbon monoxide or converting carbon monoxide to carbon dioxide, which is more readily exchangeable with oxygen compared to carbon monoxide.

This application claims priority to U.S. Provisional Application Ser. No. 61/423,176, filed on Dec. 15, 2010. The entirety of the provisional application is incorporated herein by reference.

### FIELD

The present application relates generally to carbon monoxide poisoning and, in particular, to methods for treating carbon monoxide poisoning by tangential flow filtration (TFF) of blood.

### BACKGROUND

Carbon monoxide is a product of incomplete combustion of organic matter with insufficient oxygen supply to enable complete oxidation to carbon dioxide (CO₂) and is often produced in domestic or industrial settings by motor vehicles and other gasoline-powered tools, heaters, and cooking equipment. Carbon monoxide is colorless, odorless and tasteless, but highly toxic. It combines with hemoglobin to produce carboxyhemoglobin (HbCO), which is ineffective for delivering oxygen to bodily tissues, and causes a condition known as anoxemia. At concentrations as low as 667 ppm, carbon monoxide may cause up to 50% of the body's hemoglobin to convert to HbCO. A level of 50% HbCO may result in seizure, coma, and fatality.

Carbon monoxide poisoning is the most common type of fatal air poisoning in many countries. The most likely cause of carbon monoxide exposure in the civilian setting is home fires and leaky furnaces. In the United States, it has been estimated that more than 40,000 people per year seek medical attention for carbon monoxide poisoning, which also contributes to approximately 5600 smoke inhalation deaths each year. Treatment for carbon monoxide poisoning largely consists of administering 100% oxygen or providing hyperbaric oxygen therapy. Oxygen hastens the dissociation of carbon monoxide from HbCO, thus turning it back into oxyhemoglobin. There still exists, however, a need for methods and systems for effectively treating carbon monoxide poisoning.

### SUMMARY

One aspect of the present application relates to a method for treating carbon monoxide poisoning. The method comprises the step of passing the blood of a subject in need of such treatment through a tangential flow filtration (TFF) unit, wherein said TFF unit comprises a TFF filter comprising a gas permeable membrane having a first side and a second side, wherein the blood of the subject flows along the first side of the gas permeable membrane and is oxygenated by oxygen from the second side of the gas permeable membrane.

Another aspect of the present application relates to carbon monoxide removal system for treating carbon monoxide poisoning in a subject. The system comprises a TFF unit comprising a TFF filter comprising a gas permeable membrane, a first catheter carrying blood from the subject to the TFF unit, a second catheter carrying blood from the TFF unit back to the subject, a first pump controlling a flow rate of blood through the TFF unit; and a second pump controlling a flow at of an extraction fluid through the TFF unit, wherein the extraction fluid is enriched with oxygen.

### BRIEF DESCRIPTION OF FIGURES

FIG. 1 is a schematic showing a tangential flow filtration system for removing carbon monoxide from arterial blood.

### DETAILED DESCRIPTION

The following detailed description is presented to enable any person skilled in the art to make and use the invention. For purposes of explanation, specific nomenclature is set forth to provide a thorough understanding of the present invention. However, it will be apparent to one skilled in the art that these specific details are not required to practice the invention. Descriptions of specific applications are provided only as representative examples. Various modifications to the preferred embodiments will be readily apparent to one skilled in the art, and the general principles defined herein may be applied to other embodiments and applications without departing from the scope of the invention.

In case of conflict, the present specification, including definitions, will control. Following long-standing patent law convention, the terms “a”, “an” and “the” mean “one or more” when used in this application, including in the claims.

Unless otherwise indicated, all numbers expressing quantities of ingredients, reaction conditions, and so forth used in the specification and claims are to be understood as being modified in all instances by the term “about”. Accordingly, unless indicated to the contrary, the numerical parameters set forth in this specification and attached claims are approximations that can vary depending upon the desired properties sought to be obtained by the presently disclosed subject matter. As used herein, the term “about,” when referring to a value or to an amount of mass, weight, time, volume, concentration or percentage is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed method.

Unless defined otherwise, all technical and scientific terms used herein have the same meaning as commonly understood to one of ordinary skill in the art to which the presently disclosed subject matter belongs. Although any methods, devices, and materials similar or equivalent to those described herein can be used in the practice or testing of the presently disclosed subject matter, representative methods, devices, and materials are now described.

One aspect of the present application relates to a system and method for treating carbon monoxide poisoning. The method comprises passing the blood of a subject in need thereof through a filter comprising one or more gas permeable membranes to remove carbon monoxide from the blood and oxygenate hemoglobin therefrom by tangential flow filtration (TFF) against an extraction fluid, which can be a gaseous fluid or liquid.

In one embodiment, a method for treating carbon monoxide poisoning comprises passing the blood of a subject in need of such treatment through a tangential flow filtration (TFF) unit. The TFF unit comprises a TFF filter having a gas'permeable membrane. The blood of the subject flows along one side of the gas permeable membrane while the other side of the membrane is filled with an oxygen rich extraction fluid. The oxygen molecules in the extraction fluid diffuse through the gas permeable membrane and enter the blood stream to facilitate dissociation of carbon monoxide from hemoglobin and re-oxygenate the blood. The dissociated carbon monoxide molecules diffuse through the gas permeable membrane and are removed from the blood. In certain embodiments, the extraction fluid is a gaseous fluid such as pure oxygen or a gas mixture with a high oxygen content. In other embodiments, the extraction fluid is a liquid with a high oxygen content. In other embodiments, the blood of the patient is exposed to light to promote dissociation of carbon monoxide from hemoglobin. In yet other embodiments, the blood of the patient is exposed to a catalytic agent that catalyzes the conversion of carbon monoxide to carbon dioxide.

### The TFF Unit

Filtration is a pressure driven separation process that uses filters to separate components in a liquid solution or suspension based on their size differences. Filtration can be broken down into two different operational modes-normal flow filtration (NFF) and tangential flow filtration (TFF). In NFF, fluid is converted directly toward the membrane under an applied pressure. Particulates that are too large to pass through the pores of the filter accumulate at the membrane surface or in the depth of the filtration media, while smaller molecules pass through to the downstream side. This type of process is also called dead-end filtration.

In TFF, the fluid is pumped tangentially along the surface of the filter. The fluid may be a liquid or gas. An applied pressure serves to force a portion of the fluid through the filter to the filtrate side. As in NFF, particulates and macromolecules that are too large to pass through the filter pores are retained on the upstream side. In this case the retained components do not build up at the surface of the filter. Instead, they are swept along by the tangential flow. TFF is also commonly called cross-flow filtration. The term “tangential” is descriptive of the direction of fluid flow relative to the filter.

The TFF filter can be any filter suitable for tangential flow filtration (TFF) and can include one or more filter membranes as further described herein. In one embodiment, the filter comprises a hollow fiber filter comprising a bundle of gas permeable membranes, each filter membrane being shaped in the form of a hollow tube. The feed stream (i.e., CO-containing blood) is pumped into the lumen of the tubes such that a gaseous filtrate, which contains CO released from the blood, passes through the filter membrane to the shell side, where it is removed. Alternatively, the feed stream (i.e., CO-containing blood) is pumped through the space between the hollow tubes such that a gaseous filtrate, which contains CO released from the blood, passes through the filter membrane into the lumen of the hollow tubes, where it is removed.

The dissociated carbon monoxide molecules diffuse through the gas permeable membrane and enter the extraction fluid. In certain embodiments, the TFF filter is coated with a catalytic agent that catalyzes the conversion of carbon monoxide to carbon dioxide. Examples of such catalytic agent include, but are not limited to, RuO₂(110), Pt/CeO₂—Zr O₂—Bi₂O₃ and Pd—Cu—Cl₍ₓ₎/Al₂O₃.

A gas permeable membrane may be formed from a single layer film or a multi-layer or composite film. In addition, the gas permeable membrane may be liquid impermeable. In one embodiment, the filter comprises a single layer of a gas permeable, liquid impermeable membrane. Preferably, the filter comprises a hydrophobic, gas permeable, liquid impermeable membrane. In another embodiment, the filter comprises a multi-layer membrane comprising a gas permeable film and a porous support. In a preferred embodiment, the filter is transparent to light between 300 to 700 nm. In other embodiments, the filter membrane or gas permeable membrane has a pore size that is large enough to allow exchange of gases but small enough to retain blood cells and other plasma components.

In certain embodiments, the extraction fluid flows through a filter in a direction opposite that of the blood flow to facilitate exchange of carbon monoxide with oxygen via the filter. In another embodiment, the extraction fluid flows through a hollow fiber filter in the same direction as the blood flow.

The gas permeable membrane is highly permeable to carbon monoxide and oxygen. In certain embodiments, the membrane has a permeability to oxygen and/or carbon monoxide of at least 200 barrers, preferably at least 500 barrers. Typically, the carbon monoxide/oxygen selectivity is determined by the polymer composition of the membrane.

In other embodiments, the membrane is selected to be sufficiently hydrophobic to reduce or inhibit flow therethrough so as to better retain blood components, including red blood cells, white blood cells and platelets, and plasma proteins such as albumin, immunoglobulins, fibrinogens, lipoproteins, and other regulatory proteins such as lymphokines and cytokines, inside the lumen of the membrane. In one embodiment, a hydrophobic, gas permeable membrane comprises a molecular weight cut off value of less than about 30,000 daltons, less than about 10,000 daltons, less than about 3,000 daltons, less than about 1,000 daltons, less than about 300 daltons, or less than about 100 daltons.

Preferably, the gas permeable membrane and/or fluid impermeable support layer is selected to have physical, chemical and mechanical properties such that the surface tension of the blood or plasma prevents passage of fluids therefrom through the pores of the membrane, while allowing passage of gas molecules therethrough. In some embodiments, the filter membrane or gas permeable membrane has an average pore size no greater than 1 μm, no greater than 0.1 μm, no greater than 0.01 μm, no greater than 0.005 μm, or no greater than 0.001 μm.

The porous, nonporous and/or gas permeable membranes of the present invention may be formed from a variety of polymeric materials known in the art. Exemplary polymeric membrane materials may include polyolefins, such as polyethylene and polypropylene, polyvinylidene fluoride, polyvinylidene chloride (sold commercially as SARAN™ wrap), porous TEFLON™, polysulfone, polyethersulfone, polyetherimide, polyacrylonitrile, polyimides, polyphenyleneoxide, cellulose and its derivatives, such as cellulose acetates, ethylcellulose, combinations of polymers, including polymer blends, copolymers, terpolymers and others also can be employed, as well as plastics, rubbers, such as silicone rubbers, and high temperature polyesters.

In one embodiment, the gas permeable membrane and/or liquid-impermeable membrane comprises a copolymer of perfluoro-2,2-dimethyl-1,3-dioxole (“PDD”). In another embodiment, the gas permeable membrane comprises polypropylene. In another embodiment, the gas permeable membrane comprises polypyrrolone. In yet another embodiment, the gas permeable membrane comprises a carbonized polypyrrolone.

In one embodiment, the gas permeable membrane comprises a non-porous film of a gas permeable substance. In one embodiment, the gas permeable substance is an amorphous copolymer of a perfluorinated dioxole monomer, such as PDD, as described in U.S. Pat. No. 5,876,604. In another embodiment, the copolymer formed from PDD and at least one monomer selected from the group consisting of tetrafluoroethylene (“TFE”), perfluoromethyl vinyl ether, vinylidene fluoride and chlorotrifluoroethylene. In another embodiment, the copolymer is a dipolymer of PDD and a complementary amount of TFE, especially such a polymer containing 50-95 mole % of PDD. In one embodiment, the gas permeable substance is a PDD dioxole perfluoropolymer. Other suitable polymers include, but are not limited to, polypropylene, polypyrrolone and carbonized polypyrrolone.

The present system for removing carbon monoxide may employ filters other than hollow fiber filters. In one embodiment, the filter comprises a flat plate (or cassette or capsule) module comprising layers of membrane, with or without alternating layers of separator screen, stacked together and sealed in a package. Feed fluid is pumped into alternating channels at one end of the stack and the filtrate passes through the membrane into the filtrate channels.

In another embodiment, the filter comprises a spiral wound module comprising alternating layers of membrane and separator screen wound around a hollow central core. In this case, the feed stream is pumped into one end and flows down the axis of the cartridge, whereby filtrate passes through the membrane and spirals to the core, where it is removed.

The shape of the gas permeable membrane can be a flat sheet or other geometric configuration. A flat sheet can comprise one or more monolithic films of the non-porous, gas permeable substance. Gas flux through a permeable membrane is inversely proportional to the thickness and directly proportional to the gas transport area of the membrane. One of skill in the art will readily appreciate that to obtain a practically acceptable gas flux through a gas permeable film of reasonable surface area, a very thin film should be used. This is true even though the permeability of many commercially significant gases through the amorphous copolymer preferred for use in this invention is quite high. The preferred non-porous film thickness for desirable gas flux is about 0.01 to about 25 μm.

Polymer film of less than about 12 μm generally is non-self supporting. Thus, in one embodiment, the gas permeable membrane structure comprises an amorphous copolymer present as a non-porous layer on a microporous substrate. The substrate maintains structural integrity of the non-porous layer in service. The structure of the substrate should be designed to have porosity so as not to impede the flow of the gaseous component. Representative porous substrates include a perforated sheet; a porous mesh fabric; a monolithic microporous polymer film; a microporous, hollow fiber and a combination of them.

In one embodiment, the gas permeable membrane have a tubular configuration. In a preferred embodiment, the membrane is in the form of a hollow fiber. The term “hollow fiber” refers to high aspect ratio bodies with extremely small cross section dimensions. By “high aspect ratio” refers to the ratio of the fiber length to fiber cross section dimension. Although other hollow shapes are possible and are contemplated to fall within the breadth of the present application, cylindrical hollow fibers are preferred. The fiber outer and inner diameter generally is about 0.1-1 mm and about 0.05-0.8 mm, respectively.

The separation process of this invention basically is carried out by flowing the carbon monoxide containing blood through the filter surface area of the gas permeable membrane and allowing carbon monoxide to permeate through the membrane. The term “filter surface area” means the effective area available for gas (i.e., carbon monoxide) transport. Generally, the filter surface area is the gas transport area of the membrane measured normal to the direction of gas flow. For example, the filter surface area of a rectangular flat sheet membrane is the product of sheet length and width. Similarly, the filter surface area of a single, cylindrical hollow fiber is the product of the fiber length and the circumference of the cylinder.

The preference for hollow fiber derives from the ability to create a large filter surface area in a small volume, and especially, in a volume of small overall cross sectional area. The filter surface area of a hollow fiber per unit of fiber volume increases inversely with the diameter of the fiber. Thus, surface area density of individual small diameter hollow fibers is very great. Additionally, a large number of fibers can be bundled substantially parallel to the axis of fiber elongation and manifolded. This effectively pools the filter surface area to the total of the bundled individual fiber filter surface areas. Due to the fiber geometry, a total effective filter surface area of a hollow fiber bundle can be many multiples of the overall cross sectional area of the gas filter unit. Hollow fibers also are preferred because their surface areas contact blood more effectively. That is, blood flow can be directed through bundled hollow fibers in the fiber axial direction in a way that the blood sweeps across all of the available gas filter area. In contrast, a gas filter based upon flat sheet filter elements can have poorly purged “dead spaces” of blood.

In one embodiment, the gas permeable membrane comprises a non-porous layer of amorphous copolymer that is continuous over the entire filter surface area of the membrane. That is, the non-porous layer is coextensive with the substrate and uninterrupted, being substantially free of voids, perforations or other channels which could provide open passageways through the membrane for gaseous communication between the blood side and permeate side of the membrane filter other than by permeation. Preferably, the non-porous layer is on the blood side of the gas permeable membrane. It can be appreciated that the non-porous layer presents an uncompromised barrier to penetration of blood component and plasma proteins into the micropores of the membrane. Further, the non-porous layer barrier also prevents the blood components and plasma proteins from being embedded in the microporous or ultraporous substrate and thus provides a high flux filter that resists clogging.

The non-porous gas permeable layer is located adjacent or directly on the microporous or ultraporous substrate and may be manufactured by any of a variety of methods known to those skilled in the art, including coating techniques such as dipping, spraying, painting and screeding. In one embodiment, the non-porous gas permeable layer will be applied by a solvent coating method. In some embodiments, the non-porous gas permeable layer has the thickness of about 0.01 μm to about 10 μm, about 0.1 μm to about 10 μm, about 0.1 μm to about 1 μm, or about 1 μm to about 10 μm. The non-porous gas permeable layer is well suited for processing blood which contains cells and other proteins that tend to adhere to many substrate materials. While porous membranes such as PDD copolymer membranes might resist adhesion and for a time maintain good gas flow in blood filtration, cells or plasma proteins can accumulate in the pores to eventually block flow. Moreover, if cells or other fouling occurs on the gas permeable filter surface of the non-porous layer, the surface can be cleaned easily to restore performance. The hydrophobic nature of the membrane also helps to retain the blood components, such as red blood cells, white blood cells and platelets, and plasma proteins such as albumin, immunoglobulins, fibrinogens, lipoproteins, and other regulatory proteins such as lymphokines and cytokines, inside the lumen of the membrane.

The microporous substrate pore size for use in the gas permeable membrane with a non-porous gas permeable film takes into account the molecular weight of the gas permeable polymer and the desired gas flux of the product membrane structure. Once the gas permeable polymer is selected, the molecular size of the polymer in solution can be identified. For example, PDD copolymer typically has a molecular weight of about 600,000. A microporous filter having a molecular weight cut off (“MWCO”) value of about 50,000, for example, would thus effectively filter a solution of this PDD copolymer. According to The Filter Spectrum, published by Osmonics, Inc., Minnetonka, Minn. the nominal size of an approximately 50,000 molecular weight polymer molecule on the Saccharide Type number scale is about 0.02-0.03 μm.

In certain embodiments, the microporous substrate comprises a nanofiber. Examples of nanofiber include, but are not limited to, cellulose nanofibers, biodegradable nanofibers and carbon nanofibers.

Cellulose nanofibers may be obtained from various sources such as flax bast fibers, hemp fibers, kraft pulp, and rutabaga, by chemical treatments followed by innovative mechanical techniques. The nanofibers thus obtained have diameters between 5 and 60 nm. The ultrastructure of cellulose nanofibers is investigated by atomic force microscopy and transmission electron microscopy. The cellulose nanofibers are also characterized in terms of crystallinity. In one embodiment, the membrane filter is a reinforced composite film comprising 90% polyvinyl alcohol and 10% nanofibers.

Biodegradable polymers, such as poly(glycolic acid) (PGA), poly(L-lactic acid) (PLLA) and poly(lactic-co-glycolic acid) (PLGA), can be dissolved individually in the proper solvents and then subjected to electrospinning process to make nanofibrous scaffolds. Their surfaces can then be chemically modified using oxygen plasma treatment and in situ grafting of hydrophilic acrylic acid (AA). In one embodiment, the biodegradable nanofibrous scaffold has a fiber thickness in the range of 200-800 nm, a pore size in the range of 0.5-2 μm, and porosity in the range of 94-96%.

The ultimate tensile strength of PGA will be about 2.5 MPa on average and that of PLGA and PLLA will be less than 2 MPa. The elongation-at-break will be 100-130% for the three nanofibrous scaffolds. When the surface properties of AA-grafted scaffolds are examined, higher ratios of oxygen to carbon, lower contact angles and the presence of carboxylic (—COOH) groups are identified.

Carbon nanofibers (CNF) can be synthesized by chemical vapor deposition (CVD). Amino acids, such as alanine, aspartic acid, glutamic acid and enzymes such as glucose oxidase (GOx) can be adsorbed on CNF. The properties of CNF (hydrophilic or hydrophobic) are characterized by the pH value, the concentration of acidic/basic sites and by naphthalene adsorption.

### Extraction Fluids

In the carbon monoxide removal system of the present application, at least one of the extraction fluid is enriched with oxygen. The extraction fluid can be in the form of a liquid or gas. One of the important characteristics of the extraction fluid is its ability to carry significant concentrations of oxygen. The higher the oxygen content of the extraction fluid, the greater the oxygen gradient that would drive the oxygen from the extraction side of TFF filter to the blood side of TFF filter.

In one embodiment, the extraction fluid comprises oxygen gas substantially free of liquid. Preferably, the gas comprises a sufficiently high partial pressure of oxygen to facilitate exchange of bound carbon monoxide for oxygen.

In one embodiment, the extraction fluid is 100% oxygen. In other embodiments, the extraction fluid is a gas mixture containing 50%, 60%, 70%, 80%, 90%, 95% or 99% (v/v) oxygen. In another embodiment, the extraction fluid comprises oxygen at a partial oxygen pressure of at least 160 mmHg. In yet another embodiment, the extraction fluid comprises oxygen at a partial oxygen pressure of at least 320 mmHg. In yet another embodiment, the extraction fluid is pure oxygen and the gas exchange between the blood and the extraction fluid is performed under a pressure of 2 atm, 5, atm, 10 atm or 20 atm.

In another embodiment, the extraction fluid comprises an oxygen carrier in a liquid. In certain embodiments, the extraction fluid comprises an oxygen carrier having a low affinity for oxygen. As used herein, the term low affinity for oxygen means having a P50 greater than 28 mmHg. In some embodiments, the oxygen carrier having a low affinity for oxygen comprises at least one member selected from the group consisting of pyridoxylated hemoglobin, intra molecularly cross linked hemoglobin, polymerized pyridoxylated hemoglobin, polymerized intra molecularly cross linked hemoglobin. Other oxygen carriers include, but are not limited to perfluoro chemicals, perfluoro carbon emulsions, synthetic porphyrins, and the like.

The oxygen carrier may comprise a hemoglobin preparation from human or mammalian sources. Exemplary non human hemoglobin sources include, but are not limited to, bovine, porcine or equine. In some embodiments, the oxygen carrier is an hemoglobin having a P50 of 28 mmHg or greater. In one embodiment the oxygen carrier is bovine hemoglobin (P50 of 35).

### Tangential Flow Filtration System

The carbon monoxide is removed from the blood by tangential flow filtration. Briefly, blood is removed from the patient through a first needle in an artery or a vein and is circulated by tubing through a tangential flow filtration unit comprising a gas permeable TFF filter membrane configured to remove carbon monoxide from the blood.

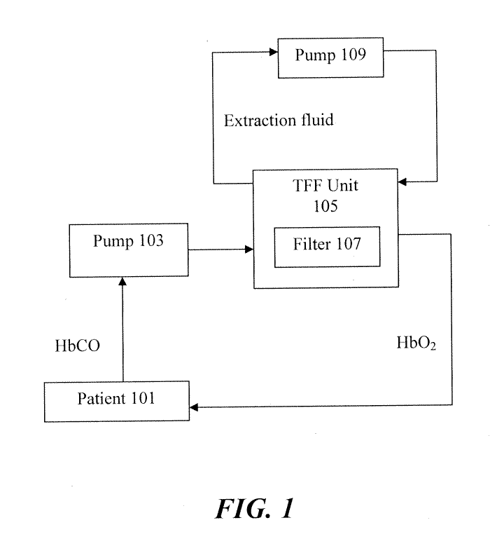

In one embodiment, exemplified in FIG. 1, the carbon monoxide-containing blood (HbCO) from patient 101 is drawn from a first catheter (not shown) and pumped through a TFF unit 105 (e.g., a filter unit having a hollow fiber filter with a molecular weight out off of less than 30,000 daltons) by a first pump 103. In the TFF unit 105, the blood flows through one side of a gas permeable TFF filter 107, while the other side of the TFF filter is exposed to an oxygen enriched extraction fluid that extracts carbon monoxide from the blood via a concentration gradient and/or a pressure gradient, and oxygenate the blood. The oxygenated blood (HbO₂) is returned to the patient 101 through a second catheter (not shown). In this embodiment, the extraction fluid is also circulated on the other side of the TFF filter by a second pump 109. The extraction fluid may be circulated through a carbon monoxide removal unit to remove the carbon monoxide collected from HbCO.

In one embodiment, the extraction fluid comprises pure oxygen to provide a reverse oxygen gradient to promote dissociation of carbon monoxide from hemoglobin. In another embodiment, the extraction fluid comprises an oxygenated liquid. The extraction in either case, the oxygenated blood (HbO₂) is then returned to the patient through a second catheter. In one embodiment, the extraction fluid flows through the TFF filter in a direction opposite that of the blood flow. In another embodiment, the extraction fluid flows through the TFF filter in the same direction as the blood flow.

In some embodiments, the gas permeable membrane has a molecular weight cut off of 30,000 dalton. In other embodiments, the gas permeable membrane has a molecular weight cut off of 10,000 dalton.

In addition to removal of carbon monoxide through exchange with oxygen as described above, carbon monoxide may be additionally removed in a number of different ways, which may be incorporated individually or in combination with the TFF system described herein. In one embodiment, carbon monoxide is removed by initially passing the carbon monoxide-containing blood through a separate TFF filter, such as a hollow fiber filter, in a low pressure environment that promote dissociation of carbon monoxide from hemoglobin. Such a low pressure environment may be created in a vacuum chamber having a pressure of less than 100 mmHg, 20 mmHg, 5 mmHg or 1 mmHg. Following removal or release of the carbon monoxide, the carbon monoxide depleted blood is subsequently oxygenated by passage through a second TFF filter against the extraction fluid as described above. In a related embodiment, the oxygenation step is performed on a second TFF filter in a pressure chamber wherein dissociation of carbon monoxide from hemoglobin and association of oxygen to hemoglobin are enhanced by oxygen pressure. In certain embodiments, the pressure chamber is filled with pure oxygen at a pressure of 5 atm, 10 atm or 20 atm.

In another embodiment, removal of carbon monoxide is facilitated by photo-dissociation of HbCO in blood. In one embodiment, dissociation of carbon monoxide from hemoglobin is accelerated by exposing the blood to a light source at 300 nm to 700 nm, preferably 400 nm to 600 nm. Examples of the light source include an incandescent light, halogen lamp, a light emitting diode, a sodium vapor lamp, or a metal halide lamp. The luminance of a single light source can be in the range of 100 to 1,000 Lm, 1,000 to 10,000 Lm, 10,000 to 100,000 Lm, or 100,000-1,000,000 Lm. In addition, two or more light sources each having the luminance mentioned above may be used in combination. This can be done by exposing the filter membrane to a halogen lamp. The photodissociation step may be selectively performed in one part of the filter or an independent filter, prior to oxygenation in the absence of the light source.

In another embodiment, carbon monoxide is removed by passing the blood through at least a portion of filter membrane coated with a transition metal having affinity for carbon monoxide, such as nickel as described in Lee et al., Desalination, 233:32-39, 2008. Preferably, the transition metal is in ionic form having a substantially higher affinity for carbon monoxide than oxygen. Transition metals for use in the present invention include, but are not limited to, nickel, iron, cobalt, copper, zinc, manganese, molybdenum, chromium, vanadium, titanium, cadmium, ruthenium, rhodium, palladium, osmium, iridium, silver, and scandium. Carbon monoxide has a particularly strong affinity for nickel. The primary requisite of the transition metal is that it forms a complex and has a first valence state in which the transition metal complex binds carbon monoxide. Often, the transition metal will also have a second valence state in which the transition metal complex is substantially inert to binding carbon monoxide. A preferred transition metal will have a valence state supporting binding to carbon monoxide, which is a substantially inert valence state for oxygen.

Transition metal containing coatings may be applied to any surface contacted with blood, including the filter membranes, catheters etc. Preferably, the blood is exposed to transition metal coated surfaces prior to oxygenation, which is performed under transition metal free conditions in view of oxygen's affinity for transition metals. By way of example, blood may be initially passed through nickel-coated catheter and/or a nickel-coated first filter for carbon monoxide removal and then oxygenated in a second nickel-free filter. Alternatively, the blood may be passed through a single filter comprising a nickel-coated region proximal to a nickel-free filter region for oxygenation.

In some embodiments, the transition metal coating will include one or more transition metal complexes or mixtures thereof wherein the complexes contain a transition metal ion and a multidentate organic chelate suitable for binding carbon monoxide. The transition metal complexes may be coordination complexes of any of a variety of transition metals as described above.

The transition metal complexes can be formed from a variety of multidentate (multicoordinate) organic chelates, especially tetradentate and pentadentate chelates, but also including tridentate and hexadentate chelates. Multidentate organic chelates may be macrocyclic, branched or linear. Suitable multidentate chelates for complexation with a transition metal may include, but are not limited to, porphyrins; porphyrin derivatives; linear, cyclic, and macrocyclic polyalkylamines; phthalocyanines and their derivatives; crown ethers; macrocyclic amines and lacunates; and Schiff base chelates, such as acetylacetonates, salicylidene amidates, and salicylidene amines. Porphyrin chelates which may be used in the transition metal complexes include naturally-occurring porphyrins, such as protoporphyrins, deuteroporphyrins, etioporphyrins, mesoporphyrins, and protoporphyrins, and synthetic porphyrins, such as tetraphenyl porphyrins and octaethyl porphyrins.

The transition metal complexes may additionally or alternatively comprise various monodentate, bidentate, and tridentate chelates including nitrogenous bases such as ammonia; primary, secondary, and tertiary amines; amino acids and their derivatives; diamines such as ethylene- and propylene-diamine; heterocyclic amines such as imidazoles, pyrroles, pyridines, bipyridyls and indoles; sulfur-containing moieties such as thiophenes, thiobenzenes, and mercaptans; phosphorus derivatives; halides and pseudohalides including chloride, bromide, iodide, cyanate, thiocyanate, cyanide, and thiocyanide; and other small molecules.

The monodentate, bidentate, and tridentate chelates will normally be combined with other chelating molecules in order to form multidentate carrier compounds. Thus, not all ligating molecules in the multidentate structure need to be covalently bound to one another. Exemplary transition metal complexes include cobalt pentaamine, a well known synthetic binding complex, where individual (monodentate) ammonia molecules are coordinated with the cobalt molecule. Combinations of bidentate and tridentate ligating molecules may also find use in forming pentadentate chelates.

Polyalkylamines are one class of multidentate chelates which may be used in the present invention. Useful polyalkylamines include linear and pentadentate, and at least four of the atoms available for coordination to the selected transition metal ion (generally referred to herein as “ligating atoms”) will be nitrogen.

In a further embodiment, carbon monoxide removal may be augmented by using a filter membrane or catheter coated with a carbon monoxide oxidation catalyst, such as a transition metal oxide, to convert the carbon monoxide to a species, such as carbon dioxide, which is more readily exchangeable with oxygen compared to carbon monoxide. Exemplary carbon monoxide oxidation catalysts include transition metal oxides, including, but not limited to oxides of manganese, copper, nickel, and mixtures of two or more of these metals. These oxides and mixtures can also be used in combination with oxides of silver, iron, tin, and various other metals. Other metals that serve as catalysts for the oxidation of carbon monoxide include indium and bismuth, which may be used either alone or in combination with one or more transition metal oxides. Of the transition metal oxides, mixtures of manganese dioxide and cupric oxide are preferred. These mixtures are available as a variety of commercial products. One class of products is designated by the name CARULITE™, including CARULITE 150, CARULITE 200, CARULITE 300, and others.

In a preferred embodiment, the carbon monoxide oxidation catalyst comprises a nitrided transition metal oxide nanoscale particles or nanoscale oxynitrides represented by the general formula MₓO_{y}N_{z} (x>0; y>0; z>0) where M represents at least one transition metal selected from the group consisting of Sc, Ti, V, Cr, Mn, Fe, Co, Ni, and Cu; and where O is oxygen and N is nitrogen. Nanoscale particles of this type can facilitate conversion of carbon monoxide to carbon dioxide in the presence or absence of oxygen.

More particularly, a nanoscale oxynitride having the formula MₓO_{y}N_{z} (y>x) is referred to as an oxygen-rich or Type A oxynitride. Type A oxynitride clusters can undergo a geometric distortion upon initial adsorption of a CO molecule. This distortion can occur in the presence or absence of an external source of oxygen. The distortion involves the breaking of a metal-oxygen bond via the adsorption of a CO molecule. The metal-oxygen bond scission creates an unsaturated oxygen atom in a favorable path of access for a subsequent CO molecule. The subsequent CO molecule can be oxidized by the unsaturated oxygen atom. A Type A cluster can oxidize CO to CO₂ by donating a lattice oxygen from the cluster. Thus, in the reaction between a Type A cluster and CO the Type A cluster can be reduced to form a Type B cluster.

A nanoscale oxynitride having the formula M_{X}O_{Y}N_{Z} (y≦x) is referred to as an oxygen-poor or Type B oxynitride. An example of a Type B cluster in the iron oxide system is Fe₂ON. The ground state geometry of a Fe₂ON cluster is a distorted rhombus. In the presence of an external source of oxygen, Type B clusters such as Fe₂ON can adsorb CO molecules and, via the formation of a CO₃ intermediate, desorb a CO₂ molecule. The reaction between a Type B cluster and CO can oxidize the Type B cluster to form a Type A cluster. The initial CO adsorption by a Type A cluster can form active catalytic sites within the cluster that can be continuously regenerated to sustain catalytic conversion and/or oxidation of carbon monoxide. Furthermore, in the absence of an external source of oxygen a Type B clusters can adsorb a CO molecule.

In one embodiment, the blood is exposed to carbon monoxide oxidation catalyst-coated surfaces prior to oxygenation, which is performed under high oxygen conditions. Thus, blood may be initially passed through a first filter coated with carbon monoxide oxidation catalyst for carbon monoxide conversion and then oxygenated in a second filter subjected to high oxygen conditions. Alternatively, the blood may be passed through a single filter comprising a carbon monoxide oxidation catalyst-coated region proximal to an uncoated region for oxygenation.

In another embodiment, filter membrane surfaces and/or catheters are coated with transition metals for removing carbon monoxide and transitional metal oxides for converting carbon monoxide to carbon dioxide.

### Carbon Monoxide Removal System

In a further aspect, the present invention provides a system for treating carbon monoxide poisoning in a subject. In one embodiment, the system comprises a TFF unit comprising a gas permeable, liquid impermeable TFF filter; a first catheter carrying blood from the subject to the TFF unit; a second catheter carrying blood from the TFF unit back to the subject; a first pump controlling a blood flow rate through the TFF unit; and a second pump controlling a flow rate of an extraction fluid through the TFF unit. These pumps may be used to control and/or monitor the fluid flow rates, transmembrane pressure, blood pressure, carbon monoxide and/or oxygen levels, and electrolyte concentrations in the blood.

In one embodiment, the system further comprises one or more monitors monitoring blood pressure, blood oxygen pressure, and/or blood carbon monoxide pressure.

In another embodiment, the TFF filter is a hollow fiber filter configured to remove carbon monoxide.

In another embodiment, the system further comprises a heating unit (blood warmer) or heat exchanger to maintain the temperature of the blood.

In another embodiment, the TFF unit comprises a first TFF filter located in a vacuum chamber and a second TFF filter located in a pressure chamber.

In yet another embodiment, the system comprises a light source suitable for illuminating blood prior to the TFF process.

In the carbon monoxide removal system of the present invention, any catheter surface, filter membrane surface, or other blood exposed surface may be coated with one or more transition metals for removing carbon monoxide from blood hemoglobin as described above. In addition, any catheter surface, filter membrane surface, or other blood exposed surface may be coated with one or more carbon monoxide oxidation catalysts for converting carbon monoxide in blood hemoglobin to carbon dioxide as described above.

The blood flow rate is preferably optimized for effective separation of carbon monoxide while maintaining a shear force that does not adversely affect the blood components. For example, the shear force should not cause the lysis of red blood cells or activate platelets. As used herein, the term “blood flow rate” refers to the rate the blood flows through the filtration unit. In one embodiment, the blood flow rate is in the range of 100-5000 ml/min.

In one embodiment, the extraction fluid flows through the filter in a direction opposite to the flow direction of the blood. In another embodiment, the extraction fluid flows through the filter in the same direction as the blood.

In another embodiment, the extraction fluid flows through the filter at a flow rate that is two to three times that of the blood flow rate.

The present invention is further illustrated by the following examples which should not be construed as limiting. The contents of all references, patents and published patent applications cited throughout this application, as well as the Figures and Tables are incorporated herein by reference.

### Example 1

### Proof of Concept Study was Undertaken in a Baboon

The proof of concept study was undertaken in a baboon. Baboon's blood has an oxygen carrying capacity, as well as oxygen loading and unloading (P50) characteristics, identical to those of humans. The animal was anesthetized under sterile conditions and intubated to permit the regulation of ventilation. Arterial and venous access was obtained. The animal was then given a mix of oxygen and carbon monoxide to convert approximately 80% of its red blood cell hemoglobin to carboxyhemoglobin. This functionally reduces the oxygen carrying capacity of the animal by 80%. The remaining 20% functional hemoglobin is adequate to maintain normal body functions in this experimental setting.

A exchange transfusion was undertaken, where 50 mL of the baboon blood was withdrawn through the artery and simultaneously replaced with an equal volume of a polymerized hemoglobin solution. This isovolumic exchange was continued until a polymerized hemoglobin concentration of approximately 6 gm/dL was achieved in the plasma phase. This process took approximately 15 minutes.

A sample of whole blood was withdrawn from the arterial line for analysis. 50% (or 3 gm/dL) of the infused hemoglobin in the plasma phase had converted to carboxyhemoglobin, while the same amount of hemoglobin within the red blood cells (or 3 gm/dl) had converted to oxyhemoglobin, thereby providing a permanent remediation of the carbon monoxide poisoning. This exchange can be carried out to provide the oxygen carrying capacity deemed necessary.

This experiment suggests that it is possible to reduce the amount of carboxyhemoglobin within the red blood cells by exposing the carboxyhemoglobin to an exogenous hemoglobin oxygen carrier. Such exposure can be achieved without exposing the patient to the hemoglobin oxygen carrier in a TFF setting. Specifically, the TFF device contains a filter having a gas permeable membrane that permits ready passage of oxygen and carbon monoxide but will not permit the passage of hemoglobin or any of its possible chemical modifications. The exogenous hemoglobin oxygen carrier is an hemoglobin oxygen carrier having a P50 of 28 torr or greater. The hemoglobin can be of human or non human source such as bovine, equine or porcine. Hemoglobin oxygen carriers with a high P50 (i.e., a low affinity to oxygen), such as bovine hemoglobin (P50 of 35), is preferable. The gas permeable membrane has a molecular weight cut off of 30,000 dalton and preferably has a molecular weight cut off of 10,000 dalton.

The above description is for the purpose of teaching the person of ordinary skill in the art how to practice the present invention, and it is not intended to detail all those obvious modifications and variations of it which will become apparent to the skilled worker upon reading the description. It is intended, however, that all such obvious modifications and variations be included within the scope of the present invention, which is defined by the following claims. The claims are intended to cover the claimed components and steps in any sequence which is effective to meet the objectives there intended, unless the context specifically indicates the contrary.

What is claimed is:

1. A method for treating carbon monoxide poisoning, comprising:
passing the blood of a subject in need thereof through a tangential flow filtration (TFF) unit, wherein said TFF unit comprises a TFF filter comprising a gas permeable membrane having a first side and a second side, wherein said blood of said subject flows along said first side of said gas permeable membrane and is oxygenated by oxygen from said second side of said gas permeable membrane.

2. The method of claim 1, wherein said gas permeable membrane has a molecular weight cut off value of 30,000 dalton or smaller.

3. The method of claim 1, wherein said gas permeable membrane is impermeable to liquid.

4. The method of claim 1, wherein said blood of said subject is oxygenated by an extraction fluid having an oxygen content (v/v) that is higher than the oxygen content (v/v) of said blood.

5. The method of claim 4, wherein the extraction fluid is an oxygen gas substantially free of liquid.

6. The method of claim 4, wherein the extraction fluid is 100% oxygen.

7. The method of claim 4, wherein the extraction fluid is a liquid saturated with oxygen.

8. The method of claim 7, wherein said extraction fluid has a partial oxygen pressure of at least 160 mmHg.

9. The method of claim 7, wherein said extraction fluid has a partial oxygen pressure of at least 320 mmHg.

10. The method of claim 1, wherein said extraction fluid is a liquid comprising an oxygen carrier.

11. The method of claim 10, wherein said oxygen carrier is a hemoglobin based oxygen carrier.

12. The method of claim 1, wherein said hemoglobin based oxygen carrier has a P50 of, or greater than, 28 mmHg.

13. The method of claim 12, wherein said hemoglobin based oxygen carrier is boving hemoglobin.

14. The method of claim 1, wherein said gas permeable membrane is coated with a carbon monoxide oxidation catalyst.

15. The method of claim 14, wherein said carbon monoxide oxidation catalyst is a transition metal, a transition metal oxide or a mixture thereof.

16. The method of claim 1, wherein said blood flows through said TFF pre-filter under illumination of light with a wave length in the range of 400 nm to 600 nm.

17. The method of claim 1, wherein said TFF unit further comprises a TFF pre-filter for removing carbon monoxide from said blood of said subject, wherein said blood flows through said TFF pre-filter under conditions that facilitate dissociation of carbon monoxide from hemoglobin or oxidation of carbon monoxide.

18. The method of claim 17, wherein said blood flows through said TFF pre-filter is located in a vacuum chamber having a pressure of less than 5 mmHg.

19. The method of claim 17, wherein said blood flows through said TFF pre-filter under illumination of light with a wave length in the range of 400 nm to 600 nm.

20. The method of claim 17, wherein at least a portion of said TFF pre-filter is coated with a transition metal, a transition metal oxide or a mixture thereof.

21. A carbon monoxide removal system for treating carbon monoxide poisoning in a subject, comprising:
a TFF unit comprising a TFF filter comprising a gas permeable membrane;
a first catheter carrying blood from said subject to said TFF unit;
a second catheter carrying blood from said TFF unit back to said subject;
a first pump controlling a flow rate of blood through said TFF unit; and
a second pump controlling a flow rate of an extraction fluid through said TFF unit, wherein said extraction fluid is enriched with oxygen.

22. The carbon monoxide removal system of claim 21, wherein said extraction fluid is pure oxygen.

23. The carbon monoxide removal system of claim 21, wherein said extraction fluid is a liquid solution comprising an oxygen carrier.

24. The carbon monoxide removal system of claim 21, wherein said TFF filter is located in a pressure chamber.

25. The carbon monoxide removal system of claim 21, wherein said TFF unit comprises a first TFF filter located in a vacuum chamber and a second TFF filter located in a pressure chamber.
